(19) United States
(12) Patent Application Publication
(10) Pub. No.: US 2017/0296403 A1
(43) Pub. Date: Oct. 19, 2017

(54) TAMPON SYSTEM WITH DRY WICK STRING AND DISPOSABLE POUCHES

(71) Applicant: Dr. Ashton Essentials, Inc., Englewood, NJ (US)

(72) Inventors: Jennifer Ashton, Englewood, NJ (US); Jeffrey M. Cohen, Pleasanton, CA (US)

(21) Appl. No.: 15/457,391

(22) Filed: Mar. 13, 2017

Related U.S. Application Data
(60) Provisional application No. 62/307,410, filed on Mar. 11, 2016.

(51) Int. Cl. A61F 13/551; A61F 13/34; A61F 13/26; A61F 13/20

(52) U.S. Cl. CPC A61F 13/55185; A61F 13/5518; A61F 13/202; A61F 13/26; A61F 13/34

(57) ABSTRACT

A tampon system comprising a tampon and an outer package is disclosed. The tampon consists of an absorbent member, a gripping member, a non-absorbent tether connected to the absorbent member and the gripping member, and a substantially sealable pouch. The substantially sealable pouch and the tampon are contained within the outer package. The outer package may be sealed and biodegradable.

### CROSS REFERENCE TO RELATED APPLICATIONS

This application claims priority to U.S. Provisional Patent Application Ser. No. 62/307,410, filed on Mar. 11, 2016 and entitled “Tampon System with Dry Wick String and Disposable Pouches”, the disclosure of which is incorporated herein by reference.

### TECHNICAL FIELD

The present invention relates to a tampon system designed to facilitate the easy usage and disposal of the same as well as to extend product service duration.

### STATEMENT REGARDING FEDERALLY SPONSORED RESEARCH OR DEVELOPMENT

(Not applicable)

### BACKGROUND OF THE INVENTION

Even though feminine hygiene tampons have been around for over eighty years, their design has not significantly changed. Moreover, tampons that are currently available for purchase have several design flaws.

One such design flaw relates to the length of the string. Tampons that are available on the market are designed with short length strings. One end of the string is attached to the tampon itself and, during use, the other end of the string extends from the vaginal area. However, because these tampon strings are short, they may easily get misplaced inside the vaginal area of the user. This misplacement of the string may lead to the user forgetting about the tampon, which in turn may lead to the user inserting a new one and pushing the old one in further. Even if the user does not forget about the tampon, searching for the misplaced string is not hygienic and many users do not feel comfortable using their fingers to probe the vaginal area to locate said string.

Another design flaw relates to the material that is used for both the tampon and the string. Oftentimes, the tampon string will get wet while urinating, leaving a cold wet string in the outer vaginal area, making the user feel unclean and dampening the user's undergarments. This may cause the user to remove and replace a newly inserted tampon to be more hygienic. However, removing a dry tampon from a dry vagina may create painful abrasions. Further, because the string is short and wet, the user has a hard time grasping the string such that it will not slip out of the grip when the user pulls on it to try and remove the tampon.

Yet another design flaw relates to the disposal of both the soiled tampon and the soiled tampon applicator. Inserting a tampon with an applicator during active bleeding leaves the applicator itself covered in blood. Further, many tampon applicators are made of materials which cannot be disposed of by flushing them down the toilet. In addition, disposal of a soiled tampon is often messy. In situations when the soiled tampon cannot be flushed down the toilet, the user is unable to dispose of it appropriately. Current commercially available tampons do not provide a way of disposing of either the soiled tampon applicator or the soiled tampon itself.

Based on these design flaws, it is apparent that there is a need for a new tampon system which will solve these problems and provide the user with a better experience when removing or replacing soiled tampons.

The present invention relates to a biodegradable tampon system comprising a dry wick tampon with an extra long dry wick tampon string constructed with a grasping feature and disposable pouches for convenient disposal of the soiled applicator and the soiled tampon.

### SUMMARY OF THE INVENTION

The inventive tampon system comprises an outer package and a tampon. The tampon contains an absorbent member, a gripping member and an absorbent tether connected to a non-absorbent tether and a substantially sealable pouch. The tampon and the substantially sealable pouch are contained within the outer package.

The non-absorbent tether is at least the same length as the absorbent tether. Further, the non-absorbent tether may form a loop that is connected to the absorbent tether.

The tampon may also contain a non-absorbent grasping feature, which is attached to the non-absorbent tether. The grasping feature may be a bead.

The outer package of the tampon system is sealed and biodegradable.

Alternatively, the tampon system may comprise an outer package and a tampon, wherein the tampon is made of an absorbent member, a gripping member, a non-absorbent tether connected to said absorbent member and said griping member, and a substantially sealable pouch, all contained within the outer package. The non-absorbent tether may form a loop that is connected to the absorbent member.

The tampon may also contain a non-absorbent grasping feature, which is attached to the non-absorbent tether. The grasping feature may be a bead.

The outer package of the tampon system is sealed and biodegradable.

### BRIEF DESCRIPTION OF THE DRAWINGS

The operation of the inventive system will become apparent from the following description taken in conjunction with the drawings, in which:

FIG. 1 is an exploded schematic prospective view of the invention, in the form of a tampon system.

FIG. 2 is a schematic perspective view of an alternate embodiment of a grasping structure.

FIG. 3 is a schematic perspective view of a disposable pouch.

FIG. 4 is a schematic perspective view of an alternate embodiment of disposable pouch.

### DETAILED DESCRIPTION OF THE PREFERRED EMBODIMENTS

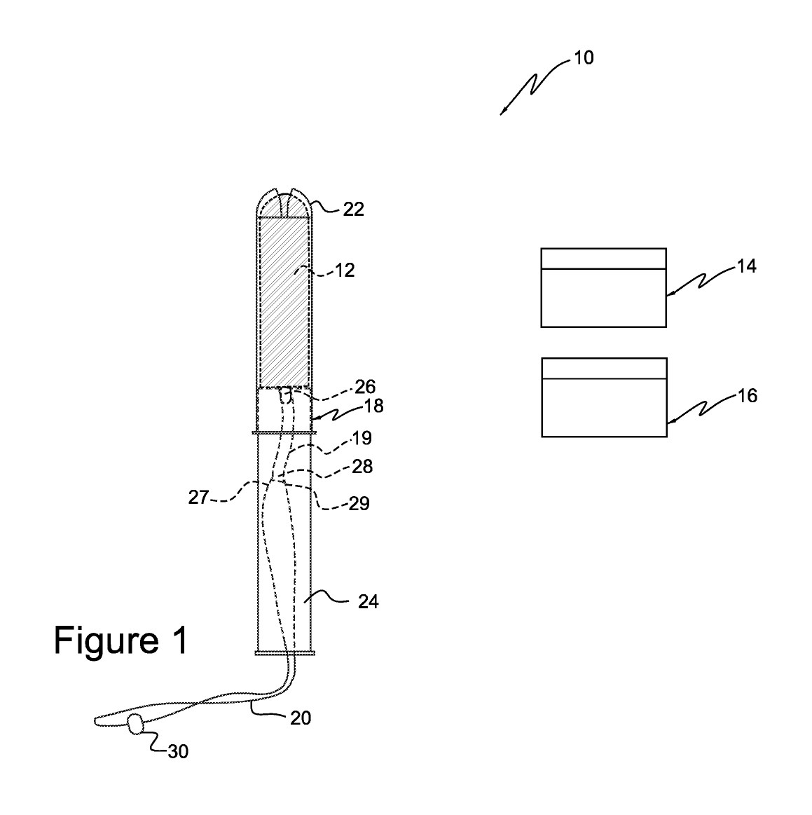

FIG. 1 is a perspective view of tampon system 10 made in accordance with the present invention comprising tampon 12, disposable pouches 14 (optionally serving as a consumer package) and 16, applicator 18, biodegradable string 19, and dry wick string 20. Applicator 18 comprises front portion 22 and a rear portion 24, where rear portion 24 is smaller in diameter than front portion 22 such that when force is exerted on rear portion 24, rear portion 24 is pushed into front portion 22, displacing and ejecting tampon 12 from front portion 22 into the vaginal cavity of the user.

String 19 comprises two ends 26 and 28. String 20 comprises two ends 27 and 29. End 26 is attached to the bottom portion of tampon 12 and end 28 is attached to ends 27 and 29. Ends 27 and 29, when attached to end 28, form a loop which comprises a grasping feature 30.

In accordance with the invention, tampon 12 and string 19 may be made from dry wick biodegradable material, such as a material of a conventional nature, whereas string 20 and grasping feature 30 may be made from non-absorbent, preferably biodegradable material, such as nylon, or plastic coated or waxed cotton string. Further, grasping feature 30 may take on many configurations that are small enough not to irritate the vaginal area of a user but are big enough to aid in grasping of string 20 by the user.

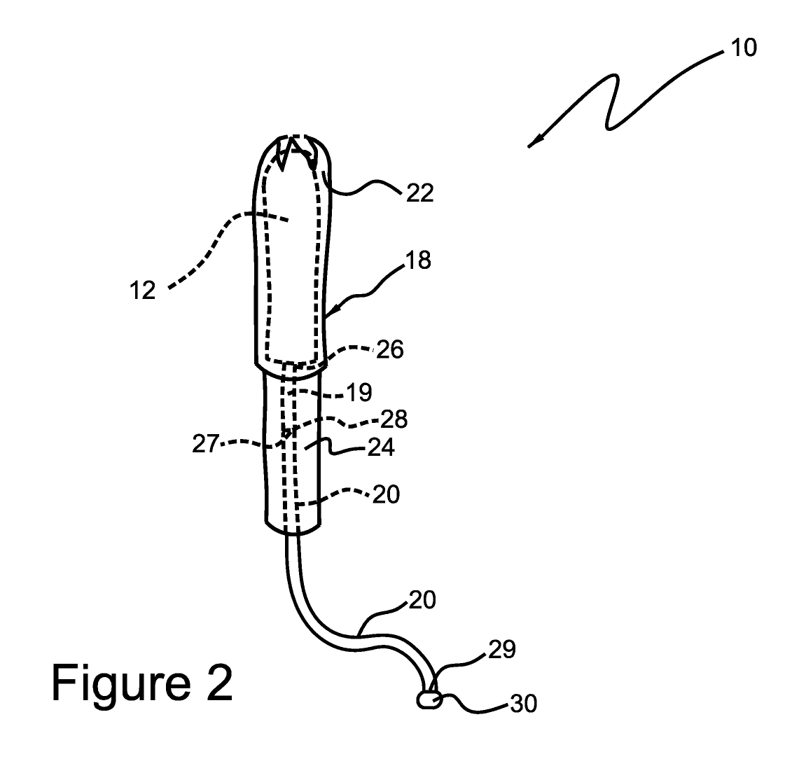

In one embodiment of the invention, as shown in FIG. 1, grasping feature 30 takes the form of a loop with a bead attached. In another embodiment of the invention, as shown in FIG. 2, grasping feature 30 takes the form of a bead. FIGS. 1 and 2 only represent two of many embodiments of the inventive grasping feature 30. Such embodiments should not be construed as limiting but rather as illustrative.

Further referring to FIG. 1, disposable pouch 16 is used for the disposal of applicator 18 and tampon 12, respectively. Disposable pouch 14 may be dimensioned and configured to house the constituents of the tampon system, namely the tampon ejector and pouch 16, thus serving as a consumer package.

Optionally, Pouch 14 may be made resealable to provide a backup disposal bag, or if pouch 14 is slightly larger than pouch 16, pouch 14 may serve as a backup pouch for disposal, or as a second pouch to contain pouch 16, thus providing a double barrier.

Pouches 14 and 16 may optionally take a form similar to conventional ziplock kitchen bags.

Optionally, a further disposable pouch may be used to dispose of applicator 18 only. Pouches 14 and 16 are optionally and desirably printed with an attractive pattern or have a native texture with an attractive appearance, for example, unbleached paper.

In the same manner, disposable pouch 16 may be dimensioned and configured to the specifications of tampon 12 such that disposable pouch 16 may be used to dispose of tampon 12 only. However, since used applicator 18, where bottom portion 24 is pushed into top portion 22, takes on roughly the same dimensions as used tampon 12, disposable pouch 16 may be used to dispose of applicator 18 and a used tampon.

Disposable pouches 14 and 16 may be formed from plastic coated paper, for example with the plastic or wax coating, for example only on the inside of pouch 14, or be made of plastic film, for example biodegradable plastic film, in an assembly procedure using suitable biodegradable and water resistant material. Alternatively, pouches 14 and 16 may be formed in multiple parts and the individual parts may be joined together to form the finished disposable pouches 14 and 16.

Pouches 14 and 16 may also be formed using a biodegradable, non-water resistant material. In such a case, to prevent disposable pouches 14 and 16 from leaking through once the soiled applicator 18 or soiled tampon 12 are placed inside, internal lining of pouches 14 and 16 may be fitted with water resistant barrier 32. Barrier 32 may be made of any suitable water resistant material. Further, outer lining of pouches 14 and 16 have a simple, elegant design that does not draw attention as to what is contained therein.

Referring to FIG. 2, a schematic perspective view of an alternate embodiment of a grasping structure is illustrated. End 28 of string 19 is tethered to string 20 at end 27, leaving end 29 a free end. Free end 29 comprises a grasping feature 30.

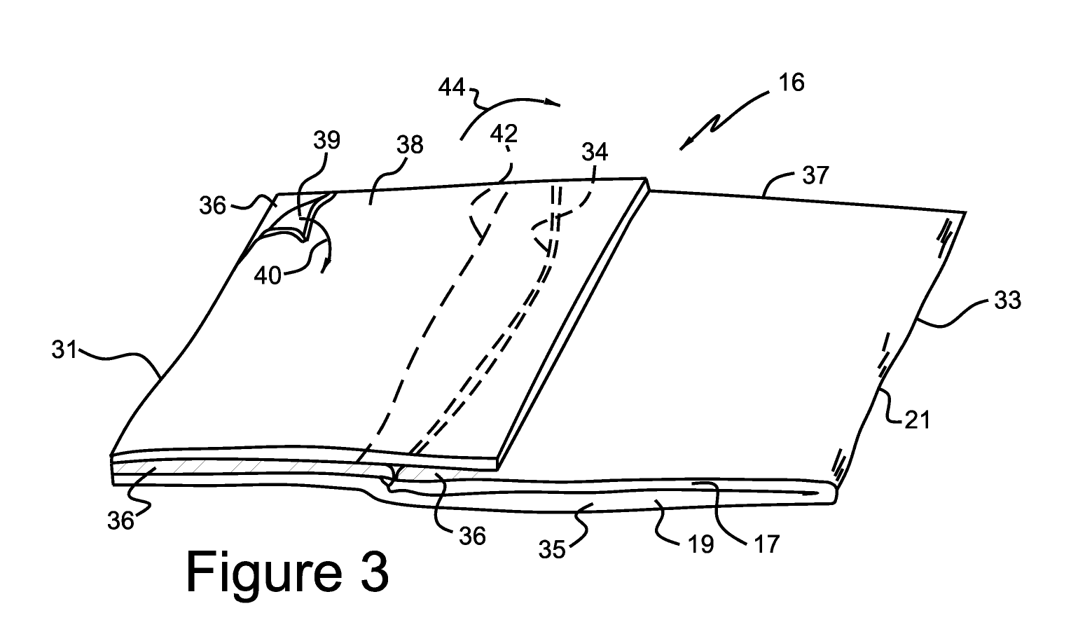

Referring to FIG. 3, a perspective view of an unsealed disposable pouch 16 is illustrated. A compartment is formed in pouch 16 between front face 17 and rear face 19. Front face 17 and rear face 19 may be formed from a single layer folded over at end 21. Disposable pouch 16 comprises a top side 31, a bottom side 33, peripheral sides 35 and 37 (where front face 17 and rear face 19 are joined to each other to form a pouch compartment for receiving a soiled tampon), an opening 34 and an adhesive layer 36. Opening 34 is configured and dimensioned to allow for easy disposal of soiled tampon into disposable pouch 16.

Adhesive layer 36 is dimensioned to cover at least the flap portion 35 of disposable pouch 16, and, optionally, continuing over and past opening 34. Adhesive layer 36 may be protected from unwanted exposure with a thin protective layer 38, for example, of paper coated with a release agent 39. Optionally, protective layer 38 may be provided in two parts, one on one side of a folding line 42, and the other on the other side of folding line 42. The release layer contacts adhesive layer 36. After, for example, tampon 12 is placed inside pouch 16, through opening 34, protective layer 38 (or both portions of protective layer 38) may be peeled, optionally, in the direction of arrow 40. Once protective layer 38 is removed, exposing adhesive layer 36, disposable pouch 16 may be folded along line 42 in the direction of arrow 44. This results in adhesive on both sides opening 34, very effectively sealing it.

Adhesive layer 36 may be configured and dimensioned to extend beyond folding line 42 such that when disposable pouch 16 is folded along folding line 42, opening 34 is sealed when adhesive layer 36 touches at least a portion of disposable pouch 16.

It should be noted that disposable pouch 14, optionally designated for disposal of a soiled applicator 18 (for example together with the last used tampon) or for use as a second pouch to for a pouch within a pouch and thus well sealed enclosure, may have the same characteristics of disposable pouch 16 as illustrated in FIG. 3.

Optionally, two pouches, having an adhesive structure like pouch 16 may be included in an optionally non-reusable consumer package.

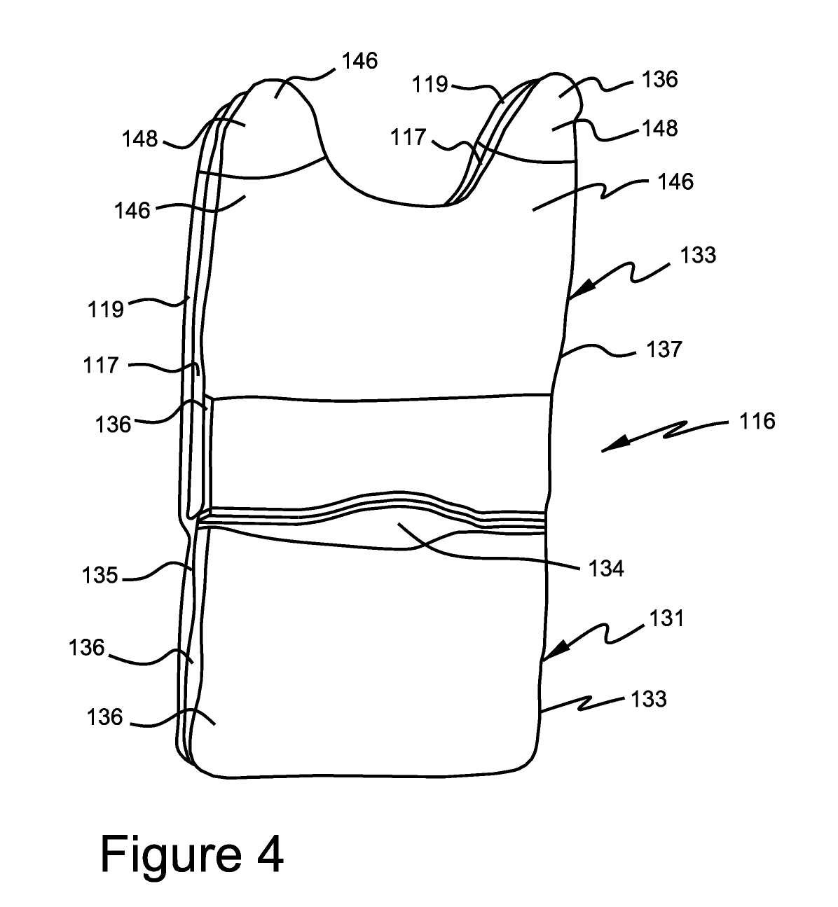

An alternate embodiment of the inventive disposable pouch 116 is illustrated in FIG. 4. Generally, similar parts or parts performing analogous, corresponding or identical functions to those of the embodiment shown in FIGS. 1 through 3, are numbered herein with numbers which differ from those of the earlier described embodiment or embodiments by multiples of one hundred.

Disposable pouch 116 comprises flap side 131, compartment side 133, and peripheral sides 135 and 137. Flap side 131 of disposable pouch 116 comprises designated compartments 146 for, for example, for the thumb and index finger. Compartments 146 and the large compartment to which they extend are formed by front and rear flexible planar layers 117 and 119, which may be made of vinyl, plastic or coated paper, or any suitable material. This allows for facile gripping and removal of a soiled tampon. Front flexible planar layer 117 is shown pulled away from rear flexible planar layer 119 to show the formation of compartments 146. Compartments 146 may be made in a single procedure of same material as disposable pouch 116. Alternatively, compartments 146 may be made in multiple parts and the individual parts may be joined together to form disposable pouch 116. For clarity of illustration, the adhesive protecting member and its release layer are not included in this view as if the adhesive protecting member has been removed.

Inside portion of flap 133 of disposable pouch 116 is covered with an adhesive layer 136 which may be covered by protective layer similar to layer 38, but not illustrated. Further, outside layer of compartments 146 may contain a rubber layer 148 or layer of any kind of material suitable to aid in the gripping process and prevent slipping of the tampon which the user is trying to grip and remove the tampon. After removal, the tampon may be enclosed by turning the flap upon itself and sealing the pouch.

Alternatively, after removal the tampon may be enclosed by turning compartment 134 inside out and by then turning the flap upon itself and sealing the pouch. However in such an embodiment, adhesive layer 136 would be deposited on the opposite side of the flap.

Further referring to FIG. 4, when a user decides to remove tampon 112 from the vaginal cavity, the user inserts her fingers into designated compartments 146. With the covered fingers, the user grips either string 120 or grasping feature 130. The user, utilizing designated compartments 146 fitted with rubber layer 148, will have a secure hold of either string 120 or grasping feature 130 (or even the tampon) such that when the user pulls string 120 or grasping feature 130, tampon 112 will be dislodged from vaginal cavity and will be removed without the user soiling her hand.

Once tampon 112 is removed from body, bottom side 133 is pulled upward, toward top side 131, closing compartments 146, effectively covering tampon 112. Once tampon 112 is covered by bottom side 133, protective layer 138 is removed, exposing adhesive layer 136. Disposable pouch 116 is sealed when exposed adhesive layer 136 overlies at least a portion of top side 131.

While illustrative embodiments of the invention have been described, it is noted that various modifications will be apparent to those of ordinary skill in the art in view of the above description and drawings. Such modifications are within the scope of the invention which is limited and defined only by the following claims.

What is claimed is:

1. A tampon system comprising an outer package, a tampon comprising an absorbent member, a gripping member, an absorbent tether, said absorbent tether connected to said absorbent member, a non-absorbent tether, said non-absorbent tether connected to said absorbent tether and said gripping member, and a substantially sealable pouch, said substantially sealable pouch and said tampon contained within said outer package.

2. A tampon system as in claim 1, wherein said non-absorbent tether is at least the same length as said absorbent tether.

3. A tampon system as in claim 1, wherein said non-absorbent tether forms a loop connected to said absorbent tether.

4. A tampon system as in claim 1 further comprising a non-absorbent grasping feature attached to said non-absorbent tether.

5. A tampon system as in claim 4, wherein said grasping feature comprises a bead.

6. A tampon system as in claim 1, wherein said outer package is sealed and biodegradable.

7. A tampon system comprising an outer package, a tampon comprising an absorbent member, a gripping member, a non-absorbent tether connected to said absorbent member and said griping member, and a substantially sealable pouch, said substantially sealable pouch and said tampon contained within said outer package.

8. A tampon system as in claim 7, wherein said non-absorbent tether forms a loop.

9. A tampon system as in claim 7 further comprising a non-absorbent grasping feature attached to said non-absorbent tether.

10. A tampon system as in claim 9, wherein said grasping feature comprises a bead.

11. A tampon system as in claim 7, wherein said outer package is sealed and biodegradable.
